(19) United States
(12) Patent Application Publication
(10) Pub. No.: US 2006/0199269 A1
(43) Pub. Date: Sep. 7, 2006

(54) Method for assessing pyrimidine metabolism

(76) Inventor: David R. Schneider, Royal Oak, MI (US)

Correspondence Address: GIFFORD, KRASS, GROH, SPRINKLE & CITKOWSKI, P.C, PO BOX 7021, TROY, MI, 48007-7021, US

(21) Appl. No.: 11/073,956

(22) Filed: Mar. 7, 2005

(51) Int. Cl. G01N 33/00

(52) U.S. Cl. 436/98

(57) ABSTRACT

A method is provided for determining pyrimidine metabolism of an individual. By collecting a saliva sample containing a concentration of a pyrimidine from an individual, the saliva sample can be measured for the pyrimidine to yield a pyrimidine measurement. Through the comparison of the pyrimidine measurement to a value measured for a control person with a known pyrimidine metabolism, the relative pyrimidine metabolism of the individual is determined. An individual with a defective or deficient ability to metabolize pyrimidines is vulnerable to dietary excess of pyrimidines or is at a great likelihood of suffering undesirable and potentially lethal side effects if the individual is given anti-cancer or anti-viral pyrimidine analogs. A kit is provided that includes a pipette and a piece of filter paper for collecting saliva from an individual. The use of saliva as a sampling substance indicative of an individual's pyrimidine metabolism affords numerous advantages over conventional usage of blood or urine.

### FIELD OF THE INVENTION

The present invention in general relates to assessing the ability of an individual to metabolize pyrimidines, and in particular to assessing pyrimidine metabolism of an individual through a saliva test.

### BACKGROUND OF THE INVENTION

5-fluorouracil and its derivatives are important anti-cancer and anti-viral chemotherapeutics. Known pyrimidine analogs having therapeutic effect include 5-fluorouracil, idoxuridine, trifluridine, bromovinyl deoxyuridine, fluorodeoxyuridine and cytarabine.

As pyrimidine analog drugs are highly effective in the treatment of cancers and serious viral infections, these compounds are well-established treatments throughout the world. A major problem associated with pyrimidine analog therapeutics is that approximately 7% of the world human population is unable to metabolize pyrimidines and as such dosing levels rapidly achieve toxic levels. In such individuals severe intestinal bleeding is regularly noted. Such individuals are known to have a defective DPD (dihydropyrimidine dehydrogenase) enzyme in the pyrimidine metabolic pathway. In addition to those individuals with an inoperative DPD enzyme, an additional approximately 5-6% of the human world population has a DPD enzyme function that is insufficient to clear pyrimidines at a normal rate and, as such, individuals being treated with a pyrimidine analog therapeutic suffer a degree of intestinal bleeding until dosage modification can be performed.

While the importance of pyrimidine metabolism differences between individuals is known to the art, previous methods measuring pyrimidine metabolism have involved isotope studies. U.S. patent application Publication US 2003/0068272A1 is representative thereof. Other techniques have involved the measurement of indirect catabolic degradation products from cerebrospinal fluid, blood plasma, or urine.

In view of the importance of identifying an individual's ability to metabolize pyrimidine analogs before beginning treatment with this class of therapeutics, there exists a need for a noninvasive and efficient test of the metabolic ability of an individual to degrade pyrimidines.

### SUMMARY OF THE INVENTION

A method is provided for determining pyrimidine metabolism of an individual. By collecting a saliva sample containing a concentration of a pyrimidine from an individual, the saliva sample can be measured for the pyrimidine to yield a pyrimidine measurement. Through the comparison of the pyrimidine measurement to a value measured for a control person with a known pyrimidine metabolism, the relative pyrimidine metabolism of the individual is determined. An individual with a defective or deficient ability to metabolize pyrimidines is vulnerable to dietary excess of pyrimidines or is at a great likelihood of suffering undesirable and potentially lethal side effects if the individual is given anti-cancer or anti-viral pyrimidine analogs. A kit is provided that includes a pipette and a piece of filter paper for collecting saliva from an individual. The use of saliva as a sampling substance indicative of an individual's pyrimidine metabolism affords numerous advantages over conventional usage of blood or urine.

### DETAILED DESCRIPTION OF THE PREFERRED EMBODIMENTS

The present invention has utility as a noninvasive method of determining proper dosing for an individual undergoing pyrimidine analog therapeutic treatment. The present invention affords pyrimidine metabolism information for an individual from saliva in contrast to prior art techniques that have relied on blood, urine, or cerebrospinal fluid in order to sample an individual's pyrimidine metabolism. The collection of saliva affords considerable advantages in requiring less skill and precaution in the collection thereof as compared to other bodily fluids. Previously, no one has identified saliva as a pyrimidine detection reservoir indicative of circulatory system levels.

Saliva collection is performed according to the present invention in any number of conventional procedures. Saliva is readily collected in a vial, with an absorbent swab, or pipetted from the buccal cavity onto an absorbent substrate. The simplicity of collecting a saliva sample allows for an untrained individual to collect such a sample. Preferably, the sample is brought into contact with a preservative so as to maintain the saliva sample integrity during transport to a measurement facility. Saliva sample collection techniques and preservatives operative herein are detailed in U.S. Pat. No. 5,968,746.

In an alternative embodiment, a kit is provided for saliva collection that includes a pipette and a piece of filter paper. The pipette is inserted into an individual's mouth and a sample of saliva collected. Optionally, a salivation stimulating substance coats the pipette tip. The saliva stimulating substance illustratively includes ascorbic acid, citric acid, and other known stimulants. With the collection of at least approximately 0.3 milliliters of saliva within the pipette, the saliva is expelled onto the filter paper. Optionally, the filter paper is treated with a substance that delineates the boundaries of a droplet of saliva thereon. A boundary delineating substance illustratively includes starch, a pH indicator, and a vegetable dye. The filter paper has a known volume saliva absorption per unit area and as such, a measurement of a pyrimidine or pyrimidine analog from a preselected unit area of filter paper correlates with concentrations in saliva of the investigated compound. While saliva dried onto filter paper is readily eluted and measured for the amount of pyrimidine or pyrimidine analog found therein, it is appreciated that passing the eluted saliva sample through a purifying column serves to enhance the signal-to-noise ratio of the subsequent measurement. Chromatography is recognized to improve measurement quality regardless of whether the sample is provided on filter paper, in a preservative containing vial, or other saliva sample collection medium. Chromatography media operative herein to isolate pyrimidine and pyrimidine analog fractions from a saliva sample illustratively include Waters XTerra® C18 or Phenomenex Aqua® C18 columns. A variety of chromatography buffers are known to successfully resolve a pyrimidine and pyrimidine analog fraction from a chromatography column. These buffers illustratively include ammonium acetate/methanol and sodium phosphate/acetonitrile.

A pyrimidine and/or pyrimidine analog fraction is measured to determine pyrimidine and/or pyrimidine analog concentration in the saliva sample. It is appreciated that dietary intake prior to pyrimidine testing according to the present invention may contribute to a pyrimidine concentration reading. For instance, pyrimidine rich foods such as Vicia faba beans eaten immediately prior to testing may in fact skew measured pyrimidine concentration. Measurement techniques conventional to the art include mass spectroscopy, capillary electrophoresis (P. Wang et al., J. Pharma & Biomed Analysis 34(2):277-283 (2004); T. Adam et al., Clin. Chem. 45:2086-2093 (1999)), nuclear magnetic resonance and optical spectroscopies. Preferably, measurement is performed by mass spectroscopy. More preferably, the mass spectrometer utilized is a quadripole or MALDI mass spectrometer. The naturally occurring pyrimidines thymine, uracil and cytosine, as well as pyrimidine analog therapeutics 5-fluorouracil, idoxuridine, trifluridine, bromovinyl deoxyuridine, fluorodeoxyuridine and cytarabine and their metabolic products, all have well known mass spectral patterns. Y. Xu et al., J. of Chromatography B, 783 (2003) 273-285; D.B. Robb et al., Anal. Chem. 72 (2000) 3653-3659; H. van Lenthe et al., Clinical Chemistry 46:12 (2000) 1916-1922; and A.B.P. van Kuilenburg et al., Clinical Chemistry 50:11 (2004) 2117-2124.

Regardless of the measurement method employed, the present invention has little difficulty discerning between an individual with normal pyrimidine metabolism and defective pyrimidine metabolism as saliva concentration difference of up to three orders of magnitude is regularly noted therebetween. Those individuals semi-competent to degrade pyrimidines provide saliva concentration levels intermediate between a normal and abnormal individual. An individual is assessed as being normal, semi-competent or abnormal with respect to pyrimidine metabolism by comparing the measurement for the individual with a measurement obtained from an individual of known pyrimidine metabolic capacity. Typically, a range of values are assembled to each of the classes of normal, semi-competent and abnormal. Information as to the competency of an individual to metabolize pyrimidines has implications in diet adjustment, vitamin intake, and responsiveness to pyrimidine analog chemotherapeutics.

While the metabolic half-life of pyrimidines in normal individuals is roughly ten minutes, an abnormal individual may have a metabolic half-life of 150 hours or more. As a result, a semi-competent or abnormal individual may still be provided with an effective anti-cancer or anti-viral pyrimidine analog provided that the metabolic half-life for the individual is taken into consideration in planning the dosing regime. Recognizing the prolonged half-life of pyrimidines in abnormal semi-competent individuals precludes bolus dosing, leading to excessive buildup of pyrimidine analogs that are associated with intestinal bleeding or neuronal damage.

In an inventive embodiment where an individual with abnormal or semi-competent pyrimidine metabolism is started on a pyrimidine analog therapeutic regimen, a second saliva sample is collected from the individual after a dose of the therapeutic pyrimidine has been administered. By noting the time between the known dosage administration and saliva collection, the metabolic kinetics for the individual are determined. With knowledge as to an individual's pyrimidine metabolic half-life, a dosing regimen is developed that prevents plasma levels of the pyrimidine analog therapeutic attaining toxic levels. Optionally, additional saliva samples are collected and measured for pyrimidine to confirm and/or adjust dosing.

### EXAMPLE

The present invention is further detailed with respect to the following non-limiting example.

Saliva samples are collected from individuals on S&S 903 filter paper (available from Schleicher & Schuell Bioscience, Inc., Keene, N.H.) The saliva sample is collected by inducing a period of salivation using a polypropylene medicine dropper coated with crystals of lemonade mix bonded to the polypropylene. The coated pipette is placed in the mouth for approximately thirty to ninety seconds causing the crystalline lemonade to dissolve and induce a pharmacologic secretory stimulus. Within the thirty to ninety second interval the individual salivates profusely. Through compressing of the medicine dropper bulb, a small aliquot of saliva is withdrawn from the tongue and place on the filter paper. The filter paper is then allowed to dry and over layered with a non-reactive coating paper for shipment to the laboratory for analysis. A small strip of the filter paper of one square centimeter is placed in a clean glass tube (10×25 mm) for analysis. Pyrimidine metabolites are removed from the filter paper by addition of 0.75 ml of 95% ethanol bringing the pyrimidine metabolites into the ethanol solution. An aliquot of the ethanol solution is introduced into a Micromass LC Quattro, a triple-quadripole mass spectrometer instrument operating in a positive electro spray ionization (ESI) mode. 5,6-dihydrouracil was noted as a multiple reaction signal for the transition m/z 115→73 as well as the transition m/z 132→115 per H. van Lenthe et al., Clinical Chemistry 46:12 (2000) 1916-1922, with this metabolite having a one-to-one correlation with uracil, fluorouracil and pyrimidine analogs thereof. Greater than an order of magnitude difference is noted between individuals who are deficient in dihydropyrimidine dehydrogenase enzyme function.

Patent documents and publications mentioned in the specification are indicative of the levels of those skilled in the art to which the invention pertains. These documents and publications are incorporated herein by reference to the same extent as if each individual document or publication was specifically and individually incorporated herein by reference.

The foregoing description is illustrative of particular embodiments of the invention, but is not meant to be a limitation upon the practice thereof. The following claims, including all equivalents thereof, are intended to define the scope of the invention.

1. A method for determining pyrimidine metabolism for an individual comprising:
collecting a saliva sample containing a concentration of a pyrimidine from an individual;
measuring said saliva sample for the pyrimidine to yield a pyrimidine measurement; and
comparing the measurement with a measured pyrimidine value for a control person with a known pyrimidine metabolism.

2. The method of claim 1 wherein measuring is by mass spectrometry.

3. The method of claim 2 wherein a quadripole or MALDI mass spectrometer is used to perform the mass spectrometry.

4. The method of claim 1 wherein said saliva sample is stored in contact with a preservative prior to measuring the pyrimidine within the saliva sample.

5. The method of claim 1 wherein the saliva sample is collected on a substrate absorbing a preselected amount of saliva.

6. The method of claim 5 wherein the substrate is filter paper.

7. The method of claim 6 wherein said filter paper further comprises an indicator.

8. The method of claim 1 further comprising passing said saliva sample through a column prior to measuring.

9. The method of claim 1 further comprising:
administering to the individual a dose of a non-naturally occurring pyrimidine analog;
waiting an amount of time for said non-naturally occurring pyrimidine analog to be metabolized;
collecting a second saliva sample; and
measuring the second saliva sample for said non-naturally occurring pyrimidine analog.

10. The method of claim 9 wherein measuring said second saliva sample is by mass spectrometry.

11. The method of claim 9 wherein said non-naturally occurring pyrimidine analog is selected from the group consisting of: 5-fluorouracil, idoxuridine, trifluridine, bromovinyl deoxyuridine, fluorodeoxyuridine and cytarabine.

12. A kit for collecting a saliva sample comprising:
a pipette suitable for oral insertion; and
a piece of filter paper having a known mass and liquid saturation volume per unit mass of said piece of filter paper.

13. The use of saliva as a sampling media to measure the pyrimidine metabolism of an individual.

14. An improved method of determining pyrimidine metabolism for an individual wherein the improvement lies in: measuring pyrimidine concentration in saliva from the individual.
